(19) United States
(12) Patent Application Publication
(10) Pub. No.: US 2026/0263051 A1
(43) Pub. Date: Sep. 10, 2026

(54) SYSTEM

(71) Applicant: SOFTBANK GROUP CORP., Tokyo (JP)

(72) Inventor: An YANAGIHARA, Tokyo (JP)

(73) Assignee: SOFTBANK GROUP CORP., Tokyo (JP)

(21) Appl. No.: 19/555,434

(22) Filed: Mar. 3, 2026

Related U.S. Application Data
(60) Provisional application No. 63/766,584, filed on Mar. 4, 2025.

(51) Int. Cl. A61B 10/00; G09B 23/28; G16H 20/00

(52) U.S. Cl. CPC A61B 10/0012; G09B 23/281; G16H 20/00; A61B 2010/0019

(57) ABSTRACT

A system for solving the challenges faced by women suffering from PMS (premenstrual syndrome) is provided. This system visualizes changes in hormone levels by having the user input information about their menstrual cycle and using generative AI to predict fluctuations in female hormones. Specifically, users input their menstrual start date, cycle length, and past symptoms via devices like smartphones or tablets. An analysis unit then predicts hormonal fluctuations based on this data. Prediction results are displayed graphically, allowing users to anticipate changes in their physical condition. Furthermore, the advice provision unit presents specific action guidelines based on the predicted fluctuations, and users can share information with others through the community unit. In this way, the system promotes understanding and countermeasures for PMS, providing support for women to better manage their health.

### CROSS-REFERENCE TO RELATED APPLICATION

This application claims priority under 35 U.S.C. § 119 to U.S. Provisional Patent Application No. 63/766,584, filed on March 4, 2025, the entire contents of which are incorporated herein by reference.

### BACKGROUND

### Technical Field

The present disclosure relates to a system.

### Related Art

Japanese Patent Application Publication Laid-Open (JP-A) No. 2022-180282 discloses a persona chatbot control method performed by at least one processor, comprising: a step of receiving a user utterance; a step of adding to the user utterance a prompt containing a description of the chatbot's persona and related instructions; a step of encoding the prompt; and a step of inputting the encoded prompt into a language model to generate a chatbot utterance in response to the user utterance.

### SUMMARY

System and method for solving two major issues faced by women suffering from PMS (premenstrual syndrome) is provided. First, there is the problem of difficulty obtaining understanding from others regarding PMS. Despite many women suffering from PMS symptoms, awareness is low, and particularly due to a lack of understanding among men, there is a current situation where appropriate consideration for PMS is difficult to obtain in the workplace and at home.

Second, it is difficult for women themselves to accurately recognize PMS. PMS symptoms are diverse and vary significantly between individuals, making it hard to connect personal physical changes to PMS and often preventing the implementation of appropriate countermeasures. The aim is to solve these problems and promote understanding and countermeasures for PMS by using generative AI to visualize fluctuations in female hormones, enabling users to more accurately grasp their own physical condition.

As a means to solve the problem, a system that visualizes female hormone fluctuations is provided. This system includes an input section for the user to input information about their menstrual cycle, enabling the user to input their menstrual start date, cycle length, and records of past symptoms. Furthermore, it includes an analysis unit containing generative AI to predict female hormone fluctuations based on the input information. Using an AI model trained on past data and typical hormone fluctuation patterns, it predicts hormone level changes for the next menstrual cycle. It also includes a visualization unit to display these predictions visually, using graphs and charts to enable intuitive understanding of hormone level fluctuations. It also includes an advice-providing section that offers users guidance to alleviate PMS symptoms, presenting specific actionable recommendations based on the predicted hormonal fluctuations. Furthermore, it incorporates a community section that provides functionality for users to share their experiences with others, facilitating the exchange of information about PMS and making it easier to gain understanding from those around them. In this way, the entire system works together to comprehensively address the challenges faced by women struggling with PMS.

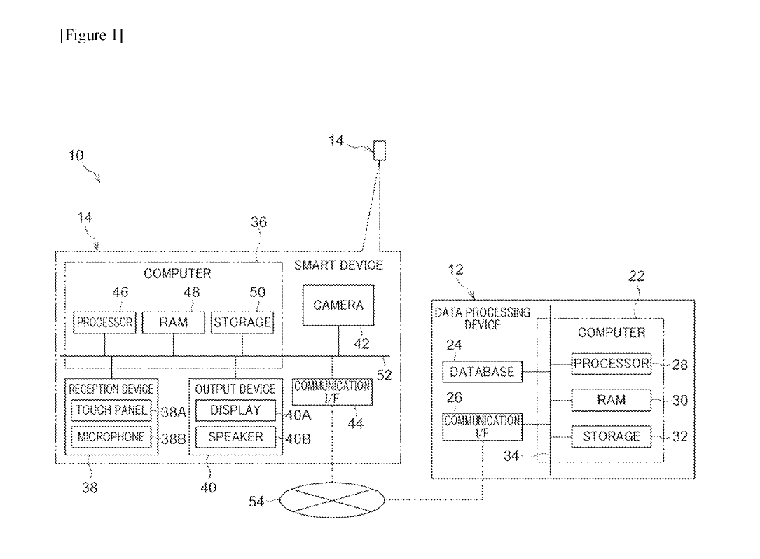

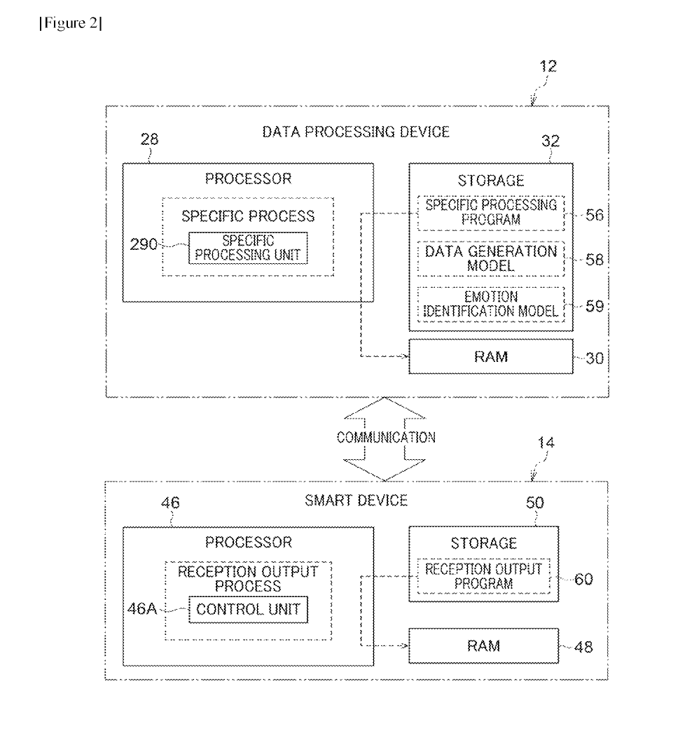

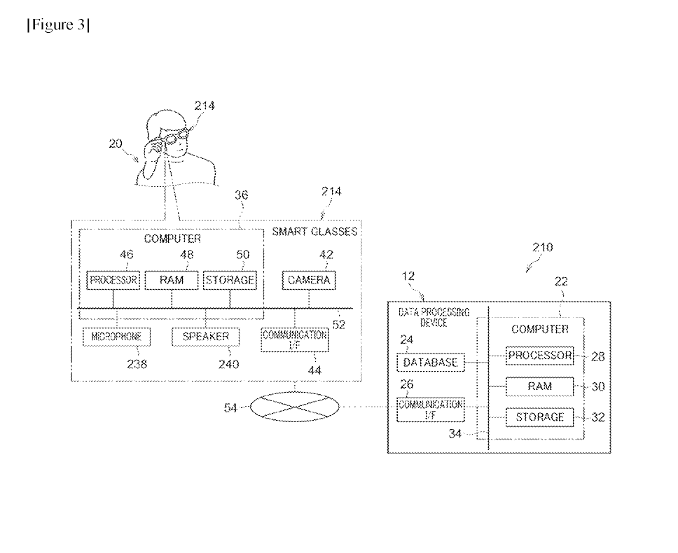

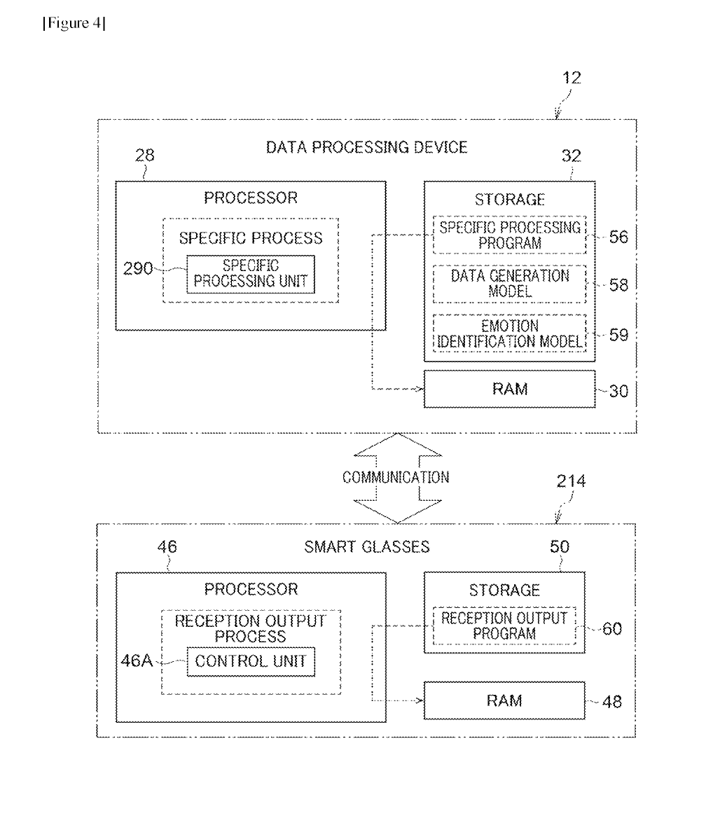

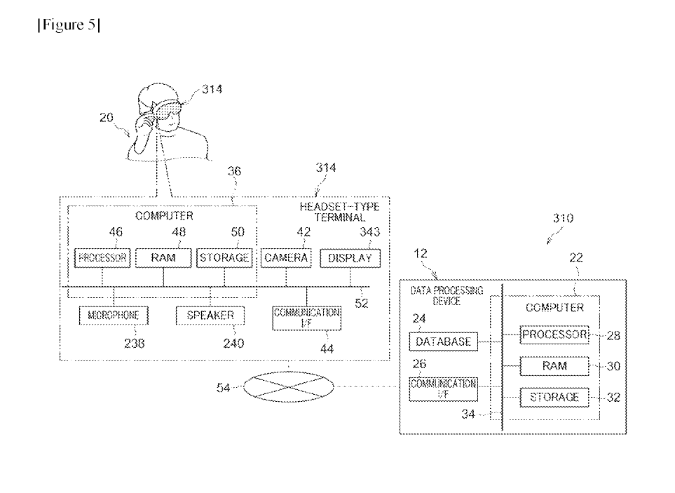

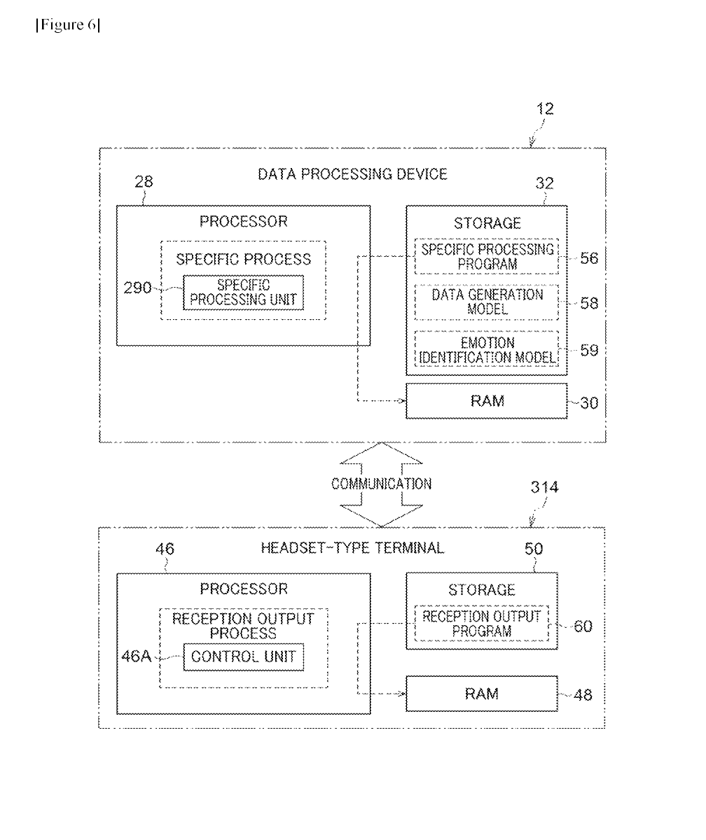

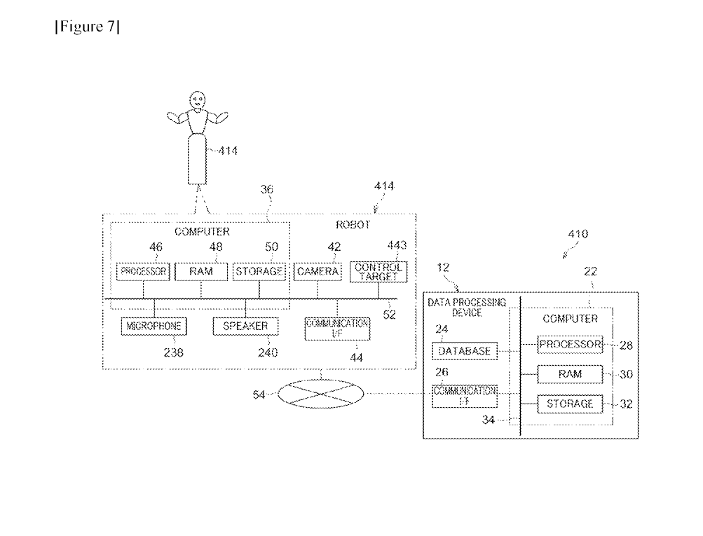

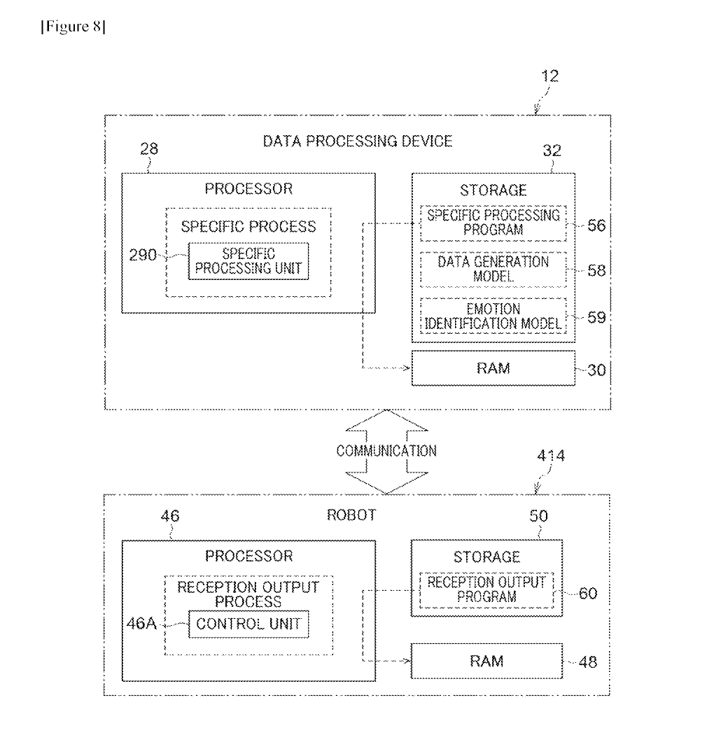

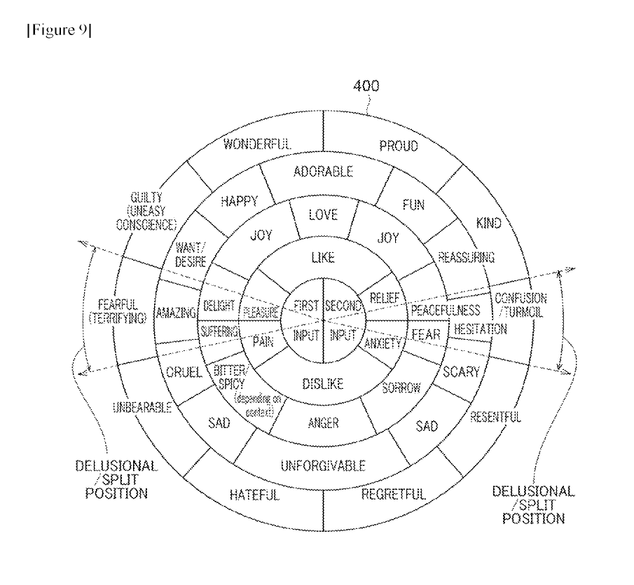

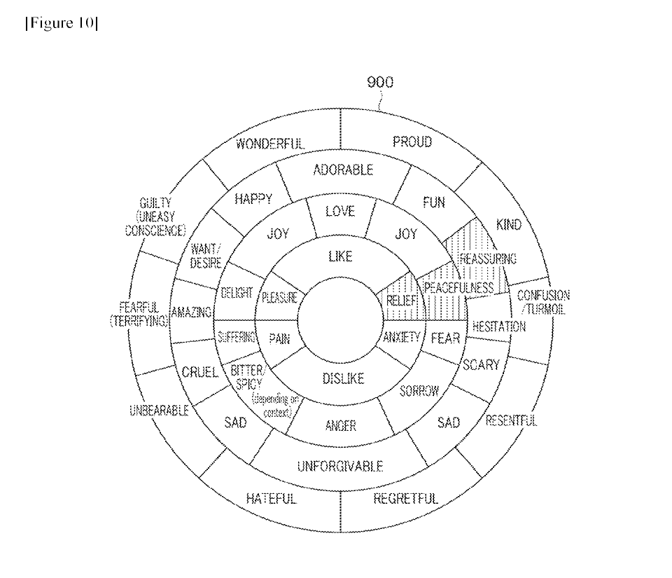

### BRIEF DESCRIPTION OF THE DRAWING

[FIG. 1] is a conceptual diagram showing an example configuration of the data processing system according to the first embodiment.

[FIG. 2] is a conceptual diagram showing an example of key functions of the data processing device and smart device according to the first embodiment.

[FIG. 3] is a conceptual diagram showing an example configuration of the data processing system according to the second embodiment.

[FIG. 4] is a conceptual diagram showing an example of the main functions of the data processing device and smart glasses according to the second embodiment.

[FIG. 5] is a conceptual diagram showing an example configuration of a data processing system according to a third embodiment.

[FIG. 6] is a conceptual diagram showing an example of the main functions of the data processing device and headset-type terminal according to the third embodiment.

[FIG. 7] is a conceptual diagram showing an example configuration of a data processing system according to the fourth embodiment.

[FIG. 8] is a conceptual diagram showing an example of the main functions of the data processing device and robot according to the fourth embodiment.

[FIG. 9] shows an emotion map onto which multiple emotions are mapped.

[FIG. 10] shows an emotion map onto which multiple emotions are mapped.

### DETAILED DESCRIPTION

The following describes an example embodiment of a system according to the present disclosure with reference to the accompanying drawings.

First, the terminology used in the following description is explained.

In the following embodiments, a processor (hereinafter simply referred to as a "processor") may be a single arithmetic unit or a combination of multiple arithmetic units. Furthermore, the processor may be a single type of arithmetic unit or a combination of multiple types of arithmetic units. Examples of processing units include a CPU (Central Processing Unit), a GPU (Graphics Processing Unit), a GPGPU (General-Purpose Computing on Graphics Processing Units), an APU (Accelerated Processing Unit), and the like.

In the following embodiments, signed RAM (Random Access Memory) is a memory where information is temporarily stored and is used as working memory by the processor.

In the following embodiments, the encoded storage is one or more non-volatile storage devices that store various programs and various parameters, etc. Examples of non-volatile storage devices include flash memory (SSD (Solid State Drive)), magnetic disks (e.g., hard disk), or magnetic tape.

In the following embodiments, the coded communication interface is an interface including a communication processor and an antenna, etc. The communication interface governs communication between multiple computers. Examples of communication standards applicable to the communication interface include wireless communication standards such as 5G (5th Generation Mobile Communication System), Wi-Fi (registered trademark), or Bluetooth (registered trademark).

In the following embodiments, "A and/or B" is synonymous with "at least one of A and B." That is, "A and/or B" means it may be only A, only B, or a combination of A and B. Furthermore, in this specification, when three or more items are expressed connected by "and/or," the same concept applies as for "A and/or B".

### First Embodiment

FIG. 1 shows an example configuration of a data processing system 10 according to the first embodiment.

As shown in FIG. 1, the data processing system 10 includes a data processing device 12 and a smart device 14. An example of the data processing device 12 is a server.

The data processing device 12 includes a computer 22, a database 24, and a communication I/F 26. The computer 22 is an example of a "computer" according to the technology of the present disclosure. The computer 22 includes a processor 28, RAM 30, and storage 32. The processor 28, RAM 30, and storage 32 are connected to a bus 34. The database 24 and communication I/F 26 are also connected to the bus 34. The communication I/F 26 is connected to a network 54. An example of the network 54 include a WAN (Wide Area Network) and/or a LAN (Local Area Network).

The smart device 14 includes a computer 36, a reception device 38, an output device 40, a camera 42, and a communication I/F 44. The computer 36 includes a processor 46, RAM 48, and storage 50. The processor 46, RAM 48, and storage 50 are connected to a bus 52. The reception device 38, output device 40, and camera 42 are also connected to the bus 52. The reception device 38, output device 40, and camera 42 are also connected to the bus 52.

The reception device 38 includes a touch panel 38A and a microphone 38B, among other components, and receives user input. The touch panel 38A receives user input via contact with an input device (e.g., a pen or finger) by detecting such contact. The microphone 38B receives voice-based user input by detecting the user's voice. The control unit 46A transmits data indicating the user input received via the touch panel 38A and microphone 38B to the data processing unit 12. Within the data processing unit 12, the specific processing unit 290 acquires the data indicating the user input.

The output device 40 includes a display 40A and a speaker 40B, among others. It presents data to the user 20 by outputting it in a form perceptible to the user 20(e.g., voice and/or text). The display 40A displays visual information such as text and images according to instructions from the processor 46. The speaker 40B outputs voice according to instructions from the processor 46. Camera 42 is a compact digital camera equipped with an optical system including a lens, aperture, and shutter, and an imaging element such as a CMOS (Complementary Metal-Oxide-Semiconductor) image sensor or a CCD (Charge Coupled Device) image sensor.

The communication interface 44 is connected to the network 54. The communication interfaces 44 and 26 manage the exchange of various information between processor 46 and processor 28 via network 54.

FIG. 2 shows an example of the main functions of the data processing device 12 and the smart device 14.

As shown in FIG. 2, specific processing is performed by processor 28 in data processing device 12. Specific processing program 56 is stored in storage 32. Specific processing program 56 is an example of a "program" related to the technology of this disclosure. Processor 28 reads the specific processing program 56 from the storage 32 and executes the read specific processing program 56 on the RAM 30. The specific processing is realized by the processor 28 operating as a specific processing unit 290 according to the specific processing program 56 executed on the RAM 30.

Storage 32 stores a data generation model 58 and an emotion identification model 59. The data generation model 58 and emotion identification model 59 are used by specific processing unit 290. Specific processing unit 290 can estimate a user's emotion using emotion identification model 59 and perform specific processing using the user's emotion. The emotion estimation function (emotion identification function) using the emotion identification model 59 performs various estimations and predictions concerning the user's emotion, including estimation and prediction of the user's emotion, but is not limited to such examples. Furthermore, estimation and prediction of emotion also includes, for example, analysis(parsing) of emotion.

The smart device 14 performs reception output processing via the processor 46. The reception output program 60 is stored in the storage 50. The reception output program 60 is used in conjunction with the specific processing program 56 by the data processing system 10. The processor 46 reads the reception output program 60 from the storage 50 and executes the read reception output program 60 on the RAM 48. The specific processing is performed by the processor 46 operating as a control unit 46A according to the specific processing program 60 executed on the RAM 48. Note that the smart device 14 may also have data generation models and emotion identification models similar to the data generation model 58 and emotion identification model 59, and may perform processing similar to that of the specific processing unit 290 using these models. The reception output processing is realized by the processor 46 operating as the control unit 46A according to the reception output program 60 executed on the RAM 48.

Other devices besides the data processing device 12 may also have the data generation model 58. For example, a server device (e.g., a generation server) may have the data generation model 58. In this case, the data processing device 12 communicates with the server device having the data generation model 58 to obtain processing results (such as prediction results) obtained using the data generation model 58. Furthermore, the data processing device 12 may be the server device itself, or it may be a terminal device owned by a user (e.g., a mobile phone, robot, home appliance, etc.). Next, an example of processing by the data processing system 10 according to the first embodiment will be described.

### Example 1

The flow of the specific processing in Example 1 is described below. The components of the system described below are implemented by the data processing device 12 and the smart device 14. The data processing device 12 is referred to as the "server," and the smart device 14 is referred to as the "terminal."

### Implementation Examples for Carrying Out the Invention

The embodiment for implementing the present invention will now be described in further detail. This system is realized using a server and a terminal, and the arrangement and functions of each component will be described in detail.

First, the input unit for receiving data from the user is primarily implemented on the terminal. The terminal may be a smartphone, tablet, personal computer, etc., with a dedicated application or web browser installed on these devices. Using these devices, the user can input the start date of menstruation, cycle length, and records of past symptoms. For example, the application provides a calendar-style interface, allowing users to easily select dates with taps and record symptoms using checkboxes. Information about daily physical condition, mood, stress levels, diet, exercise habits, and other lifestyle details can also be intuitively entered using pull-down menus and sliders.

Next, the entered data is transmitted to the server in real time. The analysis unit installed on the server possesses advanced data analysis capabilities, including generative AI. It uses AI models trained on vast historical datasets to predict fluctuations in female hormones. This AI model employs machine learning algorithms, considering both the user's individual data and general hormone fluctuation patterns. Specifically, it predicts how levels of key hormones like estrogen and progesterone will change on a daily basis. For example, the AI learns the user's hormone fluctuation patterns from past data and predicts with high accuracy the peak and decline periods for the next cycle.

The predicted hormone fluctuation data is transmitted from the server to the terminal device and visually displayed on the visualization component. This visualization component uses interactive graphs and charts to enable intuitive understanding of hormone level fluctuations. For example, estrogen peak periods and progesterone decline periods are displayed with color coding, allowing users to anticipate changes in their physical condition. Furthermore, it includes a function to compare with past data, allowing users to monitor changes in their physical condition over the long term.

Furthermore, the advice provision section is implemented on the server, generating specific recommendations for users based on predicted hormonal fluctuations and PMS symptoms. This advice is sent to the device and presented to the user. For example, it recommends yoga or meditation for relaxation during periods of rising estrogen, and encourages nutritionally balanced meals during periods of declining progesterone. It also provides tips for stress management and improving sleep quality. This advice is customized to the user's lifestyle and presented as a concrete action plan.

Finally, the community section is implemented on the server, providing a platform for users to share their experiences with others. This feature allows users to exchange information anonymously and deepen their understanding of PMS. For example, they can reference other users' experiences or receive feedback on their own symptoms. Furthermore, expert advice and the latest research information are provided, ensuring users always have access to up-to-date information.

In this way, the server and terminal collaborate to realize a system that supports women struggling with PMS in better understanding their physical condition and taking appropriate measures. This system provides a comprehensive solution tailored to individual user needs, promoting understanding of PMS and facilitating effective countermeasures.

### System Configuration

The system according to this embodiment comprises an input unit, an analysis unit, a visualization unit, an advice provision unit, and a community unit. The input unit allows users to enter information about their menstrual cycle and operates on terminals such as smartphones, tablets, or personal computers. Users can input their menstrual start date, cycle length, and records of past symptoms via a dedicated application or web browser. For example, a calendar-style interface enables users to select dates with taps and record symptoms using checkboxes. Additionally, information about daily physical condition, mood, stress levels, diet, exercise habits can be intuitively entered using pull-down menus and sliders.

The analysis unit is installed on a server, receives data transmitted from the input unit, and uses generative AI to predict fluctuations in female hormones. The analysis unit employs an AI model trained on a vast historical dataset, considering both the user's individual data and general hormone fluctuation patterns. Specifically, it predicts how levels of key hormones, such as estrogen and progesterone, will change on a daily basis. For example, the AI learns the user's hormonal fluctuation patterns from past data and predicts peak and decline periods for the next cycle with high accuracy. A specific example of a prompt fed to the generative AI is: "Based on the user's menstrual cycle data from the past six months, predict the fluctuations in estrogen and progesterone fluctuations in the next cycle based on the user's menstrual cycle data from the past six months."

The visualization unit operates on the terminal and visually displays the prediction data sent from the analysis unit. The visualization unit uses interactive graphs and charts to enable intuitive understanding of hormone level fluctuations. For example, estrogen peak periods and progesterone decline periods are displayed with color coding, allowing users to anticipate changes in their physical condition. Furthermore, it includes a function to compare with past data, enabling users to monitor changes in their physical condition over the long term.

The advice provision unit, implemented on the server, generates specific advice for the user based on predicted hormone fluctuations and PMS symptoms. This advice is sent to the terminal and presented to the user. For example, it recommends relaxation activities like yoga or meditation during periods of rising estrogen, and suggests nutritionally balanced meals during periods of declining progesterone. It also provides tips for stress management and improving sleep quality. This advice is customized to the user's lifestyle and presented as a concrete action plan.

The Community section is implemented on the server, providing a platform for users to share their experiences with others. This feature allows users to exchange information anonymously and deepen their understanding of PMS. For example, they can reference other users' experiences or receive feedback on their own symptoms. Furthermore, expert advice and the latest research information are provided, ensuring users always have access to current insights. In this way, the integrated system supports women struggling with PMS by helping them better understand their physical condition and manage it appropriately.

### Implementation Steps

### Step 1: Data Input Step

Users input information about their menstrual cycle using devices such as smartphones, tablets, or personal computers. This input is performed via a dedicated application or web browser, allowing users to enter their menstrual start date, cycle length, and records of past symptoms. For example, using a calendar-style interface, users can select dates with tap operations and record symptoms using checkboxes. Additionally, lifestyle information such as daily physical condition, mood, stress level, diet, and exercise habits can be intuitively entered using pull-down menus and sliders.

### Step 2: Data Transmission Step

The entered data is transmitted to the server in real time. This transmission uses a secure communication protocol from the device to the server, ensuring accurate data transfer while protecting user privacy.

### Step 3: Data Analysis Step

The analysis unit installed on the server analyzes the received data using generative AI. The generative AI utilizes an AI model trained on a vast historical dataset to consider both the user's individual data and general hormone fluctuation patterns. Specifically, it predicts how levels of key hormones, such as estrogen and progesterone, will change on a daily basis. An example prompt fed to the generative AI could be: "Based on the user's menstrual cycle data from the past six months, predict the fluctuations in estrogen and progesterone for the next cycle."

### Step 4: Visualization Step

Prediction data sent from the analysis unit is visually displayed on the visualization unit within the terminal. The visualization unit uses interactive graphs and charts to enable intuitive understanding of hormone level fluctuations. For example, the peak period for estrogen and the decline period for progesterone are displayed with color coding, allowing users to anticipate changes in their physical condition. Furthermore, it includes a function to compare with past data, enabling users to monitor changes in their physical condition over the long term.

### Step 5: Advice Provision Step

The advice provision unit, implemented on the server, generates specific advice for the user based on predicted hormone fluctuations and PMS symptoms. This advice is sent to the terminal and presented to the user. For example, it recommends yoga or meditation for relaxation during periods of rising estrogen, and suggests nutritionally balanced meals during periods of declining progesterone. It also provides tips for stress management and improving sleep quality. This advice is customized to the user's lifestyle and presented as a concrete action plan.

### Step 6: Community Sharing Step

The community section is implemented on the server, providing a platform for users to share their experiences with others. This feature allows users to exchange information anonymously and deepen their understanding of PMS. For example, they can reference other users' experiences or receive feedback on their own symptoms. Furthermore, expert advice and the latest research information are provided, ensuring users always have access to up-to-date knowledge.

### Specific Use Cases

Consider a use case where a user utilizes the system of the present invention. The user launches a dedicated application on their smartphone and inputs information about their menstrual cycle. Specifically, they select the start date of their period using a calendar format, input the cycle length, and select past symptoms using checkboxes. Furthermore, they can input daily physical condition, mood, stress level, diet, and exercise habits using pull-down menus and sliders.

The entered data is sent to the server in real time and processed by the analysis unit on the server. The analysis unit uses generative AI to predict the user's hormonal fluctuations in the next menstrual cycle based on past data and general hormonal fluctuation patterns. A specific example of the prompt used for this generative AI is: "Based on the user's menstrual cycle data from the past six months, predict the fluctuations in estrogen and progesterone for the next cycle."

The analysis results are sent to the terminal and visually displayed by the visualization unit. Users can check the peak and decline periods of estrogen and progesterone through interactive graphs. This enables users to anticipate changes in their physical condition and make appropriate preparations.

Furthermore, the advice provision unit generates specific advice for the user based on the predicted hormone fluctuations. For example, it recommends yoga or meditation for relaxation during periods of rising estrogen, and suggests nutritionally balanced meals during periods of declining progesterone. It also provides tips for stress management and improving sleep quality. This advice is customized to the user's lifestyle and presented as a concrete action plan.

Finally, through the community section, users can share their experiences with other users. This feature allows users to exchange information anonymously and deepen their understanding of PMS. For example, they can reference other users' experiences or receive feedback on their own symptoms. Furthermore, expert advice and the latest research information are also provided, ensuring users always have access to up-to-date information. In this way, the system offers comprehensive support to help users deepen their understanding of PMS and manage it appropriately.

### Application Example 1

The flow of specific processing in Application Example 1 is described below. The components of the system described below are implemented by the data processing device 12 and the smart device 14. The data processing device 12 is referred to as the "server," and the smart device 14 is referred to as the "terminal."

### Implementation Examples for the Present Invention

The embodiment for implementing the present invention is described in further detail below. This system monitors the health status of individuals receiving care and predicts fluctuations in health status using generative AI. The system comprises an input unit, an analysis unit, a visualization unit, an advice provision unit, and a community unit, and the specific functioning of each unit is described.

First, the input unit allows users to input information about their health status and operates on terminals such as smartwatches, tablets, or personal computers. Users can input health data such as blood pressure, heart rate, body temperature, dietary content, exercise volume, and sleep duration using these devices. For example, a smartwatch can automatically record heart rate and step count, while a tablet allows manual input of dietary content and sleep duration. This enables users to easily record their daily health status. Furthermore, these devices can connect to the server via Bluetooth or Wi-Fi, enabling automatic data synchronization. This eliminates the need for users to manually transfer data.

Next, the input data is transmitted to the server in real time. This transmission uses a secure communication protocol from the device to the server, ensuring accurate data transfer while protecting user privacy. The analysis unit installed on the server analyzes the received data using generative AI. The analysis unit uses generative AI to predict fluctuations in the user's health status based on historical data and typical health condition variation patterns. The generative AI employs machine learning algorithms, considering both the user's individual data and typical health condition variation patterns. Specifically, it predicts how key health indicators, such as blood pressure and heart rate, will change on a daily basis. An example prompt fed to the generative AI might be: "Based on the user's health data from the past three months, predict fluctuations in blood pressure and heart rate for the next week."

The predicted health fluctuation data is transmitted to the caregiver's terminal and visually displayed via the visualization unit. The visualization unit employs interactive graphs and charts to enable intuitive understanding of health metric fluctuations. For example, peak blood pressure periods and heart rate decline periods are color-coded, allowing caregivers to anticipate changes in the health status of care recipients. Furthermore, it includes a function to compare with past data, enabling caregivers to monitor changes in the health status of care recipients over the long term. This allows caregivers to detect abnormal fluctuations early and respond promptly.

The advice provision unit, implemented on the server, generates specific advice for caregivers based on predicted health status fluctuations. This advice is transmitted to the terminal and presented to the caregiver. For example, during periods of rising blood pressure, it recommends relaxation techniques like breathing exercises or light exercise. During periods of declining heart rate, it suggests nutritionally balanced meals. It also provides tips for stress management and improving sleep quality. This advice is customized to the lifestyle of the care recipient and presented as a concrete action plan. Furthermore, the advice can incorporate feedback on the effectiveness of care implemented by the caregiver, reflecting this in subsequent advice.

Finally, the Community section is implemented on the server, providing a platform for caregivers to share their experiences with other caregivers. This feature allows caregivers to exchange information anonymously and deepen their understanding of caregiving. For example, they can reference other caregivers' experiences or receive feedback on their own care methods. Furthermore, expert advice and the latest research information are provided, ensuring caregivers always have access to up-to-date knowledge. This enables caregivers to improve their skills and deliver higher-quality care.

In this way, the integrated system enables better management of the health status of care recipients and provides comprehensive support for caregivers to deliver appropriate care. This system aims to improve the quality of care and enhance the quality of life for care recipients.

### System Configuration

The system according to this embodiment comprises an input unit, an analysis unit, a visualization unit, an advice provision unit, and a community unit. The input unit allows users to input information regarding their health status and operates on terminals such as smartwatches, tablets, or personal computers. Users can input health data such as blood pressure, heart rate, body temperature, dietary content, exercise volume, and sleep duration using these devices. For example, a smartwatch can automatically record heart rate and step count, while a tablet allows manual input of dietary content and sleep duration. This enables users to easily record their daily health status. Furthermore, these devices connect to a server via Bluetooth or Wi-Fi, enabling automatic data synchronization. This eliminates the need for users to manually transfer data.

The analysis unit is installed on the server. It receives data transmitted from the input unit and uses generative AI to predict fluctuations in health status. The analysis unit employs generative AI to predict changes in the user's health condition based on patterns of past data and typical health status fluctuations. The generative AI utilizes machine learning algorithms, considering both the user's individual data and general health status fluctuation patterns. Specifically, it predicts changes in the user's health condition based on patterns of past data and typical health status fluctuations. The generative AI employs machine learning algorithms, considering both the user's individual data and general health fluctuation patterns. Specifically, it predicts how key health indicators, such as blood pressure and heart rate, will change on a daily basis. An example prompt fed to the generative AI might be: "Based on the user's health data from the past three months, predict fluctuations in blood pressure and heart rate for the next week."

The visualization unit operates on the caregiver's device, visually displaying prediction data sent from the analysis unit. It uses interactive graphs and charts to enable intuitive understanding of health metric fluctuations. For example, peak blood pressure periods and heart rate decline periods are color-coded, allowing caregivers to anticipate changes in the health status of those receiving care. Furthermore, it includes a function to compare with past data, allowing caregivers to monitor changes in the health status of care recipients over the long term. This enables caregivers to detect abnormal fluctuations early and respond promptly.

The advice provision unit, implemented on the server, generates specific advice for caregivers based on predicted health status fluctuations. This advice is transmitted to the terminal and presented to the caregiver. For example, during periods of rising blood pressure, it recommends relaxation techniques like breathing exercises or light exercise. During periods of declining heart rate, it suggests nutritionally balanced meals. It also provides tips for stress management and improving sleep quality. This advice is customized to the lifestyle of the care recipient and presented as a concrete action plan. Furthermore, the advice can incorporate feedback on the effectiveness of care implemented by the caregiver, reflecting this in subsequent advice.

The Community section is implemented on the server and provides a platform for caregivers to share their experiences with other caregivers. This feature allows caregivers to exchange information anonymously and deepen their understanding of caregiving. For example, they can reference other caregivers' experiences or receive feedback on their own care methods. Furthermore, expert advice and the latest research information are also provided, ensuring caregivers always have access to up-to-date knowledge. This enables caregivers to improve their skills and deliver higher-quality care.

In this way, the integrated system enables better management of the health status of care recipients and provides comprehensive support for caregivers to deliver appropriate care. This system aims to improve the quality of care and enhance the quality of life for care recipients.

### Implementation Steps

### Step 1: Data Input Step

Users input health-related information using terminals such as smartwatches, tablets, or personal computers. This input includes health data such as blood pressure, heart rate, body temperature, dietary content, exercise volume, and sleep duration. Smartwatches automatically record heart rate and step count, while tablets allow manual input of dietary content and sleep duration. This enables users to easily record their daily health status. Furthermore, these devices connect to the server via Bluetooth or Wi-Fi, enabling automatic data synchronization.

### Step 2: Data Transmission Step

Input data is transmitted to the server in real time. This transmission uses a secure communication protocol from the device to the server using a secure communication protocol, ensuring accurate data transfer while protecting user privacy.

### Step 3: Data Analysis Step

The analysis unit installed on the server analyzes the received data using generative AI. The generative AI predicts fluctuations in the user's health status based on historical data and typical health condition fluctuation patterns. An example prompt fed to the generative AI could be: "Based on the user's health data from the past three months, predict fluctuations in blood pressure and heart rate for the next week."

### Step 4: Visualization Step

Predictive data sent from the analysis unit is visually displayed on the caregiver's terminal via the visualization unit. The visualization unit uses interactive graphs and charts to enable intuitive understanding of health metric fluctuations. Peak blood pressure periods and heart rate decline periods are color-coded for display, allowing caregivers to anticipate changes in the health status of care recipients.

### Step 5: Advice Provision Step

The advice provision unit, implemented on the server, generates specific advice for caregivers based on predicted health status fluctuations. This advice is transmitted to the terminal and presented to the caregiver. During periods of rising blood pressure, it recommends relaxation techniques like breathing exercises or light exercise. During periods of declining heart rate, it suggests nutritionally balanced meals. It also provides tips for stress management and improving sleep quality.

### Step 6: Community Sharing Step

The Community Section is implemented on the server, providing a platform for caregivers to share their experiences with other caregivers. This feature allows caregivers to exchange information anonymously and deepen their understanding of caregiving. They can reference other caregivers' experiences and receive feedback on their own care methods. Furthermore, expert advice and the latest research information are provided, ensuring caregivers always have access to up-to-date information.

### Specific Use Cases

For example, consider a care facility utilizing the present system. Care recipients wear smartwatches to record daily health data. The smartwatches automatically measure heart rate, steps taken, and calories burned. Caregivers can manually input meal details and sleep duration on the tablet. This enables comprehensive recording of the health status of the residents.

The input data is transmitted in real time to the facility's server. The analysis unit on the server uses generative AI to analyze this data and predict fluctuations in health status. A specific example of a prompt fed to the generative AI could be: "Based on the past three months of health data, predict fluctuations in blood pressure and heart rate for the next week." This prompt enables the AI to predict individual health status fluctuations with high accuracy.

The analysis results are sent to the caregiver's tablet and visually displayed by the visualization unit. Caregivers can use interactive graphs to identify peak and trough periods for blood pressure and heart rate. For example, if a period with higher-than-normal blood pressure is predicted, it is highlighted in red on the graph to alert the caregiver. This enables caregivers to implement appropriate countermeasures in advance.

Furthermore, the advice provision unit generates specific advice for caregivers based on predicted health condition fluctuations. For instance, during periods of rising blood pressure, it recommends relaxation breathing techniques or light exercise; during periods of declining heart rate, it suggests nutritionally balanced meals. It also provides tips for stress management and improving sleep quality. This advice is customized to the lifestyle of the care recipient and presented as a concrete action plan.

Finally, through the Community section, caregivers can share information with caregivers from other facilities. This feature allows caregivers to exchange information anonymously and deepen their understanding of caregiving. For example, they can reference other caregivers' experiences or receive feedback on their own care methods. Furthermore, expert advice and the latest research information are also provided, ensuring caregivers always have access to up-to-date information.

In this way, the system of the present invention provides comprehensive support to better manage the health status of care recipients and enable caregivers to provide appropriate care. This system aims to improve the quality of care and enhance the quality of life for care recipients.

The specific processing unit 290 transmits the results of the specific processing to the smart device 14. On the smart device 14, the control unit 46A instructs the output device 40 to output the results of the specific processing. The microphone 38B acquires audio indicating user input regarding the results of the specific processing. The control unit 46A transmits the audio data indicating the user input acquired by the microphone 38B to the data processing unit 12. At the data processing unit 12, the specific processing unit 290 acquires the audio data.

Data Generation Model 58 is a so-called generative AI (Artificial Intelligence). An example of a data generation model 58 is ChatGPT (registered trademark) (Internet search <URL: https://openai.com/blog/chatgpt>). Data generation model 58 is obtained by performing deep learning on a neural network. Data generation model 58 receives input of a prompt containing instructions, as well as inference data such as audio data representing sound, text data representing text, and image data (e.g., still image data or video data) representing images. The data generation model 58 infers based on the input inference data according to the instructions indicated by the prompt and outputs the inference result in one or more data formats, such as audio data, text data, and image data. The data generation model 58 includes, for example, text generation AI, image generation AI, and multimodal generation AI. Here, inference refers to, for example, analysis, classification, prediction, and/or summarization. The specific processing unit 290 performs the aforementioned specific processing while utilizing the data generation model 58. The data generation model 58 may be a fine-tuned model capable of outputting inference results from prompts that do not contain instructions. In this case, the data generation model 58 can output inference results from prompts that do not contain instructions. The data processing device 12 and the like may include multiple types of data generation models 58. The data generation model 58 includes AI other than generative AI. AI other than generative AI includes, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others. The AI may perform various processing operations, but is not limited to such examples. Furthermore, AI may be an AI agent. Also, when the processing of the aforementioned components is performed by AI, that processing may be performed in part or in whole by AI, but is not limited to such examples. Furthermore, processing performed by AI, including generative AI, may be replaced by rule-based processing, and rule-based processing may be replaced by processing performed by AI, including generative AI.

Furthermore, the processing by the data processing system 10 described above is executed by either the specific processing unit 290 of the data processing device 12 or the control unit 46A of the smart device 14, but it may also be executed by both the specific processing unit 290 of the data processing device 12 and the control unit 46A of the smart device 14. Furthermore, the specific processing unit 290 of the data processing device 12 acquires or collects information necessary for processing from the smart device 14 or external devices, etc., and the smart device 14 acquires or collects information necessary for processing from the data processing device 12 or external devices, etc.

For example, the collection unit may be implemented by the control unit 46A of the smart device 14 or the specific processing unit 290 of the data processing device 12. For example, the acquisition unit acquires step count data using the camera 42 or communication I/F 44 of the smart device 14, and this data is processed by the specific processing unit 290 of the data processing device 12. For example, the analysis unit is implemented by the specific processing unit 290 of the data processing device 12 and analyzes data from the collection unit and acquisition unit. For example, the generation unit is implemented by the specific processing unit 290 of the data processing device 12 and generates a cooking menu using a generation AI. For example, the provision unit is implemented by the output device 40 of the smart device 14 or the specific processing unit 290 of the data processing device 12 and provides the generated cooking menu to the user. The correspondence between each unit and the device or control unit is not limited to the above examples and various modifications are possible.

The above embodiment described a form where specific processing is performed by the data processing device 12, but the technology disclosed herein is not limited thereto; specific processing may also be performed by the smart device 14.

### Second Embodiment

FIG. 3 shows an example configuration of the data processing system 210 according to the second embodiment.

As shown in FIG. 3, the data processing system 210 includes a data processing device 12 and smart glasses 214. An example of the data processing device 12 is a server.

The data processing device 12 includes a computer 22, a database 24, and a communication I/F 26. The computer 22 is an example of a "computer" according to the technology of the present disclosure. The computer 22 includes a processor 28, RAM 30, and storage 32. The processor 28, RAM 30, and storage 32 are connected to a bus 34. The database 24 and communication I/F 26 are also connected to the bus 34. The communication I/F 26 is connected to a network 54. An example of the network 54 is WAN (Wide Area Network) and/or LAN (Local Area Network) are examples.

Smart glasses 214 include a computer 36, a microphone 238, a speaker 240, a camera 42, and a communication I/F 44. The computer 36 includes a processor 46, RAM 48, and storage 50. Processor 46, RAM 48, and storage 50 are connected to bus 52. Microphone 238, speaker 240, and camera 42 are also connected to bus 52.

Microphone 238 receives voice input from user 20 to accept instructions and the like. Microphone 238 captures the voice input from user 20, converts the captured voice into audio data, and outputs it to processor 46. Speaker 240 outputs audio according to instructions from processor 46.

Camera 42 is a compact digital camera equipped with an optical system, such as a lens, aperture, and shutter, and an imaging element, such as a CMOS(Complementary Metal-Oxide-Semiconductor) image sensor or a CCD(Charge Coupled Device) image sensor. It captures images of the user's surroundings (e.g., within a field of view equivalent to that of a typical healthy individual).

The communication I/F 44 is connected to the network 54. The communication I/Fs 44 and 26 manage the exchange of various information between processor 46 and processor 28 via network 54. The exchange of various information between processor 46 and processor 28 using communication I/F 44 and 26 is performed in a secure state.

FIG. 4 shows an example of the main functions of the data processing device 12 and the smart glasses 214. As shown in FIG. 4, specific processing is performed by the processor 28 in the data processing device 12. The specific processing program 56 is stored in the storage 32.

The specific processing program 56 is an example of a "program" related to the technology of this disclosure. Processor 28 reads the specific processing program 56 from storage 32 and executes the read specific processing program 56 on RAM 30. The specific processing is realized by processor 28 operating as specific processing unit 290 according to the specific processing program 56 executed on RAM 30.

Storage 32 stores a data generation model 58 and an emotion identification model 59. The data generation model 58 and emotion identification model 59 are used by specific processing unit 290. Specific processing unit 290 can estimate a user's emotion using emotion identification model 59 and perform specific processing using the user's emotion. The emotion estimation function (emotion identification function) using the emotion identification model 59 performs various estimations and predictions concerning the user's emotion, including estimation and prediction of the user's emotion, but is not limited to such examples. Furthermore, estimation and prediction of emotion also includes, for example, analysis(parsing) of emotion.

In the smart glasses 214, the processor 46 performs the reception output processing. The reception output program 60 is stored in the storage 50. The processor 46 reads the reception output program 60 from the storage 50 and executes the read reception output program 60 on the RAM 48. The reception output processing is realized by the processor 46 operating as the control unit 46A according to the reception output program 60 executed on the RAM 48. Note that the smart glasses 214 may also have a data generation model 58 and an emotion identification model 59, and can perform processing similar to that of the identification processing unit 290 using these models.

Next, the identification processing performed by the identification processing unit 290 of the data processing device 12 is described. The components of the system described below are implemented by the data processing device 12 and the smart glasses 214. In the following description, the data processing device 12 is referred to as the "server," and the smart glasses 214 are referred to as the "terminal."

### Example 1

The flow of the specific processing is the same as that described in Example 1 of the first embodiment, so the explanation is omitted.

### Application Example 1

The flow of the specific processing in Example 1 described in the above first embodiment is the same, so the explanation is omitted.

The specific processing unit 290 transmits the result of the specific processing to the smart glasses 214. In the smart glasses 214, the control unit 46A causes the speaker 240 to output the result of the specific processing. The microphone 238 acquires audio indicating user input regarding the result of the specific processing. The control unit 46A transmits the audio data indicating the user input acquired by the microphone 238 to the data processing device 12. At the data processing device 12, the specific processing unit 290 acquires the audio data.

Data Generation Model 58 is what is known as generative AI(Artificial Intelligence). An example of a data generation model 58 is ChatGPT (registered trademark) (Internet search <URL: https://openai.com/blog/chatgpt>). Data generation model 58 is obtained by performing deep learning on a neural network. Data generation model 58 receives input of a prompt containing instructions, as well as inference data such as audio data representing sound, text data representing text, and image data (e.g., still image data or video data) representing images. The data generation model 58 infers based on the input inference data according to the instructions indicated by the prompt and outputs the inference result in one or more data formats such as audio data, text data, and image data. The data generation model 58 includes, for example, text generation AI, image generation AI, and multimodal generation AI. Here, inference refers to, for example, analysis, classification, prediction, and/or summarization. The specific processing unit 290 performs the aforementioned specific processing while utilizing the data generation model 58. The data generation model 58 may be a fine-tuned model capable of outputting inference results from prompts that do not contain instructions. In this case, the data generation model 58 can output inference results from prompts that do not contain instructions. The data processing device 12 and the like may include multiple types of data generation models 58. The data generation model 58 includes AI other than generative AI. AI other than generative AI includes, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others. The AI may perform various processing operations, but is not limited to such examples. Furthermore, the AI may be an AI agent. Also, when the processing of the aforementioned components is performed by AI, that processing may be performed in part or in whole by AI, but is not limited to such examples. Furthermore, processing performed by AI, including generative AI, may be replaced by rule-based processing, and rule-based processing may be replaced by processing performed by AI, including generative AI.

Furthermore, the processing performed by the data processing system 10 described above is executed by either the specific processing unit 290 of the data processing device 12 or the control unit 46A of the smart device 14, but it may also be executed by both the specific processing unit 290 of the data processing device 12 and the control unit 46A of the smart device 14. Furthermore, the specific processing unit 290 of the data processing device 12 acquires or collects information necessary for processing from the smart device 14 or external devices, etc., and the smart device 14 acquires or collects information necessary for processing from the data processing device 12 or external devices, etc.

For example, the collection unit may be implemented by the control unit 46A of the smart device 14 or the specific processing unit 290 of the data processing device 12. For example, the acquisition unit acquires step count data using the camera 42 or communication I/F 44 of the smart device 14, and this data is processed by the specific processing unit 290 of the data processing device 12. For example, the analysis unit is implemented by the specific processing unit 290 of the data processing device 12 and analyzes data from the collection unit and acquisition unit. For example, the generation unit is implemented by the specific processing unit 290 of the data processing device 12 and generates a cooking menu using a generation AI. For example, the provision unit is implemented by the output device 40 of the smart device 14 or the specific processing unit 290 of the data processing device 12 and provides the generated cooking menu to the user. The correspondence between each unit and the device or control unit is not limited to the above examples and various modifications are possible.

The above embodiment described a form where specific processing is performed by the data processing device 12, but the technology disclosed herein is not limited thereto; specific processing may also be performed by the smart glasses 214.

### Third Embodiment

FIG. 5 shows an example configuration of the data processing system 310 according to the third embodiment.

As shown in FIG. 5, the data processing system 310 includes a data processing device 12 and a headset-type terminal 314. An example of the data processing device 12 is a server.

The data processing device 12 includes a computer 22, a database 24, and a communication I/F 26. The computer 22 is an example of a "computer" according to the technology of the present disclosure. The computer 22 includes a processor 28, RAM 30, and storage 32. The processor 28, RAM 30, and storage 32 are connected to a bus 34. The database 24 and communication I/F 26 are also connected to the bus 34. The communication I/F 26 is connected to a network 54. An example of the network 54 is WAN (Wide Area Network) and/or LAN (Local Area Network) are examples.

The headset-type terminal 314 comprises a computer 36, a microphone 238, a speaker 240, a camera 42, a communication interface 44, and a display 343. The computer 36 includes a processor 46, RAM 48, and storage 50. The processor 46, RAM 48, and storage 50 are connected to a bus 52. The microphone 238, speaker 240, camera 42, and display 343 are also connected to the bus 52.

Microphone 238 receives voice input from user 20 to accept instructions or other commands. Microphone 238 captures the voice input from user 20, converts the captured voice into audio data, and outputs it to processor 46. Speaker 240 outputs audio in accordance with instructions from processor 46.

The camera 42 is a compact digital camera equipped with an optical system, such as a lens, aperture, and shutter, and an imaging element, such as a CMOS (Complementary Metal-Oxide-Semiconductor) image sensor or a CCD (Charge Coupled Device) image sensor. It captures images of the user's surrounding (e.g., within a field of view equivalent to that of a typical healthy individual).

The communication I/F 44 is connected to the network 54. The communication I/Fs 44 and 26 manage the exchange of various information between processor 46 and processor 28 via network 54. The exchange of various information between processor 46 and processor 28 using communication I/F 44 and 26 is performed in a secure state.

FIG. 6 shows an example of the main functions of the data processing device 12 and the headset-type terminal 314. As shown in FIG. 6, specific processing is performed by the processor 28 in the data processing device 12. The specific processing program 56 is stored in the storage 32.

The specific processing program 56 is an example of a "program" related to the technology of this disclosure. The processor 28 reads the specific processing program 56 from the storage 32 and executes the read specific processing program 56 on the RAM 30. The specific processing is realized by the processor 28 operating as a specific processing unit 290 according to the specific processing program 56 executed on the RAM 30.

Storage 32 stores a data generation model 58 and an emotion identification model 59. The data generation model 58 and the emotion identification model 59 are used by the specific processing unit 290.

In the headset-type terminal 314, reception output processing is performed by the processor 46. The reception output program 60 is stored in the storage 50. Processor 46 reads the reception output program 60 from storage 50 and executes the read reception output program 60 on RAM 48. Reception output processing is achieved by processor 46 operating as control unit 46A according to the reception output program 60 executed on RAM 48.

Next, the specific processing performed by the specific processing unit 290 of the data processing device 12 is described. The various parts of the system described below are implemented by the data processing device 12 and the headset-type terminal 314. In the following description, the data processing device 12 is referred to as the "server," and the headset-type terminal 314 is referred to as the "terminal."

### Example 1

The flow of the specific processing is the same as that described in Example 1 of the first embodiment, so the description is omitted.

### Application Example 1

The flow of the specific processing in Example 1 described in the above first embodiment is the same, so the explanation is omitted.

The specific processing unit 290 transmits the result of the specific processing to the headset-type terminal 314. At the headset-type terminal 314, the control unit 46A causes the speaker 240 and the display 343 to output the result of the specific processing. The microphone 238 acquires audio indicating user input regarding the result of the specific processing. The control unit 46A transmits the audio data indicating the user input acquired by the microphone 238 to the data processing device 12. At the data processing device 12, the specific processing unit 290 acquires the audio data.

The data generation model 58 is a so-called generative AI (Artificial Intelligence). An example of the data generation model 58 is ChatGPT (registered trademark) (Internet search <URL: https://openai.com/blog/chatgpt>). Data generation model 58 is obtained by performing deep learning on a neural network. Data generation model 58 receives input prompts containing instructions, as well as inference data such as audio data representing sound, text data representing text, and image data (e.g., still image data or video data) representing images. The data generation model 58 infers based on the input inference data according to the instructions indicated by the prompt and outputs the inference result in one or more data formats, such as audio data, text data, and image data. The data generation model 58 includes, for example, text generation AI, image generation AI, and multimodal generation AI. Here, inference refers to, for example, analysis, classification, prediction, and/or summarization. The specific processing unit 290 performs the aforementioned specific processing while utilizing the data generation model 58. The data generation model 58 may be a fine-tuned model capable of outputting inference results from prompts that do not contain instructions. In this case, the data generation model 58 can output inference results from prompts that do not contain instructions. The data processing device 12 and the like may include multiple types of data generation models 58. The data generation model 58 includes AI other than generative AI. AI other than generative AI includes, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others. The AI may perform various processing operations, but is not limited to such examples. Furthermore, the AI may be an AI agent. Also, when the processing of the aforementioned components is performed by AI, that processing may be performed in part or in whole by AI, but is not limited to such examples. Furthermore, processing performed by AI, including generative AI, may be replaced by rule-based processing, and rule-based processing may be replaced by processing performed by AI, including generative AI.

Furthermore, the processing performed by the data processing system 10 described above is executed by either the specific processing unit 290 of the data processing device 12 or the control unit 46A of the smart device 14, but it may also be executed by both the specific processing unit 290 of the data processing device 12 and the control unit 46A of the smart device 14. Furthermore, the specific processing unit 290 of the data processing device 12 acquires or collects information necessary for processing from the smart device 14 or external devices, etc., and the smart device 14 acquires or collects information necessary for processing from the data processing device 12 or external devices, etc.

For example, the collection unit may be implemented by the control unit 46A of the smart device 14 or the specific processing unit 290 of the data processing device 12. For example, the acquisition unit acquires step count data using the camera 42 or communication I/F 44 of the smart device 14, and this data is processed by the specific processing unit 290 of the data processing device 12. For example, the analysis unit is implemented by the specific processing unit 290 of the data processing device 12 and analyzes data from the collection unit and acquisition unit. For example, the generation unit is implemented by the specific processing unit 290 of the data processing device 12 and generates a cooking menu using a generation AI. For example, the provision unit is implemented by the output device 40 of the smart device 14 or the specific processing unit 290 of the data processing device 12 and provides the generated cooking menu to the user. The correspondence between each unit and the device or control unit is not limited to the above examples and various modifications are possible.

The above embodiment described a form where specific processing is performed by the data processing device 12, but the technology disclosed herein is not limited thereto; specific processing may also be performed by the headset-type terminal 314.

### Fourth Embodiment

FIG. 7 shows an example configuration of the data processing system 410 according to the fourth embodiment.

As shown in FIG. 7, the data processing system 410 includes a data processing device 12 and a robot 414. An example of the data processing device 12 is a server.

The data processing device 12 includes a computer 22, a database 24, and a communication I/F 26. The computer 22 is an example of a "computer" related to the technology of this disclosure. The computer 22 includes a processor 28, RAM 30, and storage 32. The processor 28, RAM 30, and storage 32 are connected to a bus 34. The database 24 and communication I/F 26 are also connected to the bus 34. The communication I/F 26 is connected to a network 54. An example of the network 54

include a WAN (Wide Area Network) and/or a LAN (Local Area Network).

Robot 414 includes a computer 36, a microphone 238, a speaker 240, a camera 42, a communication I/F 44, and a control target 443. Computer 36 includes a processor 46, RAM 48, and storage 50. Processor 46, RAM 48, and storage 50 are connected to bus 52. Furthermore, microphone 238, speaker 240, camera 42, and controlled object 443 are also connected to bus 52.

Microphone 238 receives voice input from user 20 to accept instructions or other commands. Microphone 238 captures the voice input from user 20, converts the captured voice into audio data, and outputs it to processor 46. Speaker 240 outputs audio in accordance with instructions from processor 46.

Camera 42 is a compact digital camera equipped with an optical system comprising a lens, aperture, and shutter, and an imaging device such as a CMOS (Complementary Metal-Oxide-Semiconductor) image sensor or CCD (Charge Coupled Device) image sensor. It captures the user's surroundings (e.g., an imaging range defined by a field of view equivalent to that of a typical healthy person).

The communication I/F 44 is connected to the network 54. The communication I/Fs 44 and 26 manage the exchange of various information between processor 46 and processor 28 via network 54. The exchange of various information between processor 46 and processor 28 using communication I/F 44 and 26 is performed in a secure state.

The control target 443 includes a display device, LEDs for the eye section, and motors for driving the arms, hands, legs, etc. The posture and gestures of robot 414 are controlled by controlling the motors for the arms, hands, legs, etc. Part of the robot 414's emotions can be expressed by controlling these motors. Furthermore, the robot 414's facial expressions can also be expressed by controlling the light emission state of the LEDs in its eyes.

FIG. 8 shows an example of the main functions of the data processing device 12 and the robot 414. As shown in FIG. 8, specific processing is performed by the processor 28 in the data processing device 12. The specific processing program 56 is stored in the storage 32.

The specific processing program 56 is an example of a "program" related to the technology of this disclosure. The processor 28 reads the specific processing program 56 from the storage 32 and executes the read specific processing program 56 on the RAM 30. The specific processing is realized by the processor 28 operating as a specific processing unit 290 according to the specific processing program 56 executed on the RAM 30.

Storage 32 stores a data generation model 58 and an emotion identification model 59. The data generation model 58 and emotion identification model 59 are used by the specific processing unit 290.

In robot 414, reception output processing is performed by processor 46. Storage 50 stores a reception output program 60. Processor 46 reads the reception output program 60 from storage 50 and executes the read reception output program 60 on RAM 48. Reception output processing is realized by the processor 46 operating as a control unit 46A according to the reception output program 60 executed on RAM 48.

Next, the specific processing performed by the specific processing unit 290 of the data processing device 12 is described. The various parts of the system described below are implemented by the data processing device 12 and the robot 414. In the following description, the data processing device 12 is referred to as the "server," and the robot 414 is referred to as the "terminal."

### Example 1

The flow of the specific processing is the same as that described in Example 1 of the first embodiment, so the explanation is omitted.

### Application Example 1

The flow of the specific processing in Example 1 described in the above first embodiment is the same, so the explanation is omitted.

The specific processing unit 290 transmits the result of the specific processing to the robot 414. In the robot 414, the control unit 46A causes the speaker 240 and the control target 443 to output the result of the specific processing. The microphone 238 acquires audio indicating user input regarding the result of the specific processing. The control unit 46A transmits the audio data indicating the user input acquired by the microphone 238 to the data processing device 12. At the data processing device 12, the specific processing unit 290 acquires the audio data.

Data Generation Model 58 is what is known as generative AI (Artificial Intelligence). An example of a data generation model 58 is ChatGPT (registered trademark) (Internet search <URL: https://openai.com/blog/chatgpt>). Data generation model 58 is obtained by performing deep learning on a neural network. Data generation model 58 receives input of a prompt containing instructions, as well as inference data such as audio data representing sound, text data representing text, and image data (e.g., still image data or video data) representing images. The data generation model 58 infers based on the input inference data according to the instructions indicated by the prompt and outputs the inference result in one or more data formats, such as audio data, text data, and image data. The data generation model 58 includes, for example, text generation AI, image generation AI, and multimodal generation AI. Here, inference refers to, for example, analysis, classification, prediction, and/or summarization. The specific processing unit 290 performs the aforementioned specific processing while utilizing the data generation model 58. The data generation model 58 may be a fine-tuned model capable of outputting inference results from prompts that do not contain instructions. In this case, the data generation model 58 can output inference results from prompts that do not contain instructions. The data processing device 12, etc., includes multiple types of data generation models 58, and the data generation model 58 includes AI other than generative AI. AI other than generative AI includes, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others. The AI may perform various processing operations, but is not limited to such example. Furthermore, the AI may be an AI agent. Also, when the processing of the aforementioned parts is performed by AI, that processing may be performed in part or in whole by AI, but is not limited to such examples. Furthermore, processing performed by AI, including generative AI, may be replaced by rule-based processing, and rule-based processing may be replaced by processing performed by AI, including generative AI.

Furthermore, the processing performed by the data processing system 10 described above is executed by either the specific processing unit 290 of the data processing device 12 or the control unit 46A of the smart device 14, but it may also be executed by both the specific processing unit 290 of the data processing device 12 and the control unit 46A of the smart device 14. Furthermore, the specific processing unit 290 of the data processing device 12 acquires or collects information necessary for processing from the smart device 14 or external devices, etc., and the smart device 14 acquires or collects information necessary for processing from the data processing device 12 or external devices, etc.

For example, the collection unit may be implemented by the control unit 46A of the smart device 14 or the specific processing unit 290 of the data processing device 12. For example, the acquisition unit acquires step count data using the camera 42 or communication I/F 44 of the smart device 14, and this data is processed by the specific processing unit 290 of the data processing device 12. For example, the analysis unit is implemented by the specific processing unit 290 of the data processing device 12 and analyzes data from the collection unit and acquisition unit. For example, the generation unit is implemented by the specific processing unit 290 of the data processing device 12 and generates a cooking menu using a generation AI. For example, the provision unit is implemented by the output device 40 of the smart device 14 or the specific processing unit 290 of the data processing device 12 and provides the generated cooking menu to the user. The correspondence between each unit and the device or control unit is not limited to the above examples and various modifications are possible.

The above embodiment described a form where specific processing is performed by the data processing device 12, but the technology disclosed herein is not limited thereto; specific processing may also be performed by the robot 414.

The emotion identification model 59, functioning as an emotion engine, may determine the user's emotion according to a specific mapping. Specifically, the emotion identification model 59 may determine the user's emotion according to an emotion map (see FIG. 9), which is a specific mapping. Furthermore, the emotion identification model 59 may similarly determine the robot's emotion, and the specific processing unit 290 may perform specific processing using the robot's emotion.

FIG. 9 is a diagram showing an emotion map 400 where multiple emotions are mapped. In the emotion map 400, emotions are arranged radially in concentric circles from the center. Emotions closer to the center of the concentric circles represent more primitive states. Emotions representing states or behaviors arising from mental states are arranged further outward on the concentric circles. Emotion is a concept encompassing affect and mental states. Generally, emotions generated from reactions occurring within the brain are placed on the left side of the concentric circles. Generally, emotions induced by situational judgment are placed on the right side of the concentric circles. Generally, emotions generated from reactions occurring within the brain and also induced by situational judgment are placed in the upper and lower directions of the concentric circles. Additionally, the upper part of the concentric circle houses "pleasant" emotions, while the lower part houses "unpleasant" emotions. Thus, in Emotion Map 400, multiple emotions are mapped based on the structure from which emotions arise, with emotions that tend to occur simultaneously mapped closer together.

These emotions are distributed around the 3 o'clock position on Emotion Map 400, typically oscillating between feelings of security and anxiety. In the right half of Emotion Map 400, situational awareness takes precedence over internal sensations, resulting in a calmer impression.

The inner part of Emotion Map 400 represents the mind, while the outer part represents behavior. Therefore, the further outward one goes on Emotion Map 400, the more visible the emotion becomes (manifesting in behavior).

Here, human emotions are based on various balances, such as posture and blood sugar levels. When these balances deviate from the ideal, it indicates a state of discomfort; when they approach the ideal, it indicates a state of comfort. Similarly, for robots, automobiles, motorcycles, etc., emotions can be created based on various balances, such as posture and battery level. When these balances deviate from the ideal, it indicates a state of discomfort; when they approach the ideal, it indicates a state of comfort. The emotion map is, for example, Dr. Mitsuyoshi's Emotion Map (Based on research on speech emotion recognition and neurophysiological signal analysis of emotions, Tokushima University, Doctoral Dissertation: https://ci.nii.ac.jp/naid/500000375379). The left half of the emotion map displays emotions belonging to the "Reaction" domain, where sensory aspects predominate. The right half of the emotion map displays emotions belonging to the "situation" domain, where situational awareness is dominant.

The emotion map defines two emotions that promote learning. One is the negative emotion around the center of the "repentance" or "reflection" area on the situation side. That is, when the robot experiences negative emotions like "I never want to feel this way again" or "I don't want to be scolded anymore." The other is the positive emotion around "desire" on the reaction side. That is, when the robot feels positive emotions like "I want more" or "I want to know more."

The emotion identification model 59 inputs the user input into a pre-trained neural network, obtains emotion values corresponding to each emotion shown in the emotion map 400, and determines the user's emotion. This neural network is pre-trained based on multiple training data sets, which are combinations of user input and emotion values corresponding to each emotion shown in the emotion map 400. Furthermore, this neural network is trained such that emotions positioned close to each other, as shown in Emotion Map 900 in FIG. 10, have similar values. FIG. 10 illustrates an example where multiple emotions, such as "reassurance," "tranquility," and "confidence," have similar emotion values.

The above description primarily explains the system of the present disclosure in terms of the functions of the data processing device 12. However, the system of the present disclosure is not necessarily implemented on a server. The system of the present disclosure may be implemented as a general information processing system. For example, the present disclosure may be implemented as a software program operating on a personal computer or as an application operating on a smartphone or similar device. The method of the present disclosure may be provided to users in a SaaS (Software as a Service) format.

The above embodiments illustrated a configuration where specific processing is performed by a single computer 22. However, the technology of this disclosure is not limited thereto. Distributed processing may be performed by multiple computers, including computer 22, for specific processing. For example, data generation model 58 may be provided in an external device of data processing device 12, and said external device may generate data corresponding to input data.

The above embodiment described an example where a specific processing program 56 is stored in storage 32, but the technology disclosed herein is not limited to this. For example, the specific processing program 56 may be stored on a portable computer-readable non-volatile storage medium such as a USB (Universal Serial Bus) memory. The specific processing program 56 stored on the non-volatile storage medium may be stored on a portable USB memory or similar device. The specific processing program 56 stored on the non-volatile storage medium is installed on the computer 22 of the data processing device 12. The processor 28 executes the specific processing according to the specific processing program 56.

Alternatively, the specific processing program 56 may be stored on a storage device, such as a server, connected to the data processing device 12 via the network 54. Upon request from the data processing device 12, the specific processing program 56 may be downloaded and installed on the computer 22.

It should be noted that it is not necessary to store the entire specific processing program 56 on a storage device such as a server connected to the data processing device 12 via the network 54, or to store the entire specific processing program 56 in the storage 32. It is also possible to store only a portion of the specific processing program 56.

Various types of processors can be used as hardware resources to execute the specific processing. Examples of processors include a CPU, which is a general-purpose processor that functions as a hardware resource for executing specific processing by executing software, i.e., programs. Additionally, processors may include dedicated electronic circuits, such as FPGAs (Field-Programmable Gate Array), PLDs (Programmable Logic Device), or ASICs (Application Specific Integrated Circuit), which are processors with circuit configurations specifically designed to execute particular processing tasks. Each processor incorporates or connects to memory, and each processor executes specific processing by utilizing this memory.

The hardware resources for executing specific processing may be comprised of one of these various processors, or may be comprised of a combination of two or more processors of the same or different types(e.g., a combination of multiple FPGAs, or a combination of a CPU and an FPGA). Furthermore, the hardware resources for executing specific processing may be a single processor.

Examples of configurations using a single processor include: First, a configuration where one processor is formed by combining one or more CPUs with software, with this processor functioning as the hardware resource executing the specific processing. Second, there is a form using a processor that implements the entire system functionality, including multiple hardware resources executing specific processing, on a single IC chip, as exemplified by a System-on-a-chip(SoC). Thus, specific processing is implemented as a hardware resource using one or more of the various processors described above.

Furthermore, regarding the hardware structure of these various processors, more specifically, electrical circuits combining circuit elements such as semiconductor devices can be used. Also, the specific processing described above is merely one example. Therefore, it goes without saying that within the scope not deviating from the main purpose, unnecessary steps may be omitted, new steps may be added, or the processing order may be changed.

The above description and illustrations provide a detailed explanation of the aspects pertaining to the technology of this disclosure and represent merely one example of the technology disclosed herein. For example, the above descriptions of the configuration, functions, actions, and effects are merely examples of the configuration, functions, actions, and effects pertaining to the technology disclosed herein. Therefore, it goes without saying that within the scope that does not deviate from the spirit of the technology disclosed herein, unnecessary portions may be omitted, new elements may be added, or replacements may be made to the above-described content and illustrated content. Furthermore, to avoid confusion and facilitate understanding of the technology disclosed herein, descriptions of technical common knowledge and the like that are not particularly necessary for enabling the implementation of the technology disclosed herein have been omitted from the above-described content and illustrated content. To facilitate understanding of the technical aspects of the present disclosure, descriptions of common technical knowledge that are not particularly necessary for enabling the present disclosure have been omitted from the above descriptions and illustrations.

All literature, patent applications, and technical specifications cited herein are incorporated by reference to the same extent as if each individual literature, patent application, and technical specification were specifically and individually cited herein.

Regarding the above embodiments, the following is further disclosed.

### Supplementary Note 1

A system for monitoring health status and predicting changes in health status using generative AI, comprising: an input unit for inputting health-related information from a user; an analysis unit including generative AI for predicting health status fluctuations based on the input information; a visualization unit for visually displaying the predicted health status fluctuation data; an advice provision unit for providing advice to the user based on the health status fluctuations; and a community unit for providing a community function allowing the user to share information about their own health status with other users.

The input unit operates on a device such as a smartwatch or tablet, enabling the user to input health data such as blood pressure, heart rate, body temperature, dietary content, exercise volume, and sleep duration, and is equipped with a function to transmit this data to a server in real time, as described in Supplementary Note 1.

The analysis unit is installed on the server and uses generative AI to predict fluctuations in the user's health status on a daily basis based on past data and general health condition fluctuation patterns. It employs specific examples such as the prompt sentence "Based on the user's health data from the past three months, predict fluctuations in blood pressure and heart rate for the next week" to be fed into the generative AI. This constitutes the system described in Supplementary Note 1.

### EXPLANATION OF SYMBOLS

10, 210, 310, 410 Data Processing System

12 Data Processing Device

14 Smart Device

214 Smart Glasses

314 Headset-type devices

414 Robot

What is claimed is:

1. A menstrual cycle prediction system, comprising:
a user terminal device including a processor, a display, and an input interface configured to receive user health data;
a server device including:
at least one processor;
a non-transitory storage storing executable instructions, a transformer-based generative neural network model, and a user profile database; and
a communication interface configured to communicate with the user terminal device over a network;
wherein the user terminal device is configured to transmit to the server device menstrual-cycle–related health data formatted according to a predefined structured data schema including:
a cycle start date,
a basal body temperature value,
at least one symptom log entry selected from mood, abdominal pain, or fatigue, and
a timestamp;
wherein the server device processor is configured to:
generate a normalized time-series feature vector from the transmitted menstrual-cycle–related health data;
construct a structured prompt including the normalized time-series feature vector and stored historical cycle data retrieved from the user profile database;
input the structured prompt into the transformer-based generative neural network model to generate a predicted future hormone-level trend and a predicted next cycle start date;
generate a formatted output including a time-series forecast and a recommended action schedule associated with the predicted future hormone-level trend;
transmit the formatted output to the user terminal device for display; and
update a cycle prediction smoothing parameter stored in the user profile database using exponential smoothing based on subsequently received actual cycle start date data, thereby forming a closed-loop prediction update process.

2. The system of claim 1, wherein the normalized time-series feature vector includes a rolling seven-day average of basal body temperature values.

3. The system of claim 1, wherein the structured prompt comprises a predefined template including labeled fields corresponding to cycle phase, symptom intensity score, and temperature deviation value.

4. The system of claim 1, wherein the transformer-based generative neural network model comprises a multi-layer attention-based architecture fine-tuned using historical menstrual cycle datasets.

5. The system of claim 1, wherein the recommended action schedule includes at least one of sleep duration adjustment, dietary modification, or exercise intensity modification mapped to predicted luteal or follicular phases.

6. The system of claim 1, wherein the user terminal device is configured to:
render a graphical user interface including a plurality of visual state modes corresponding respectively to follicular, ovulatory, and luteal phases; and
automatically transition between the visual state modes based on the predicted future hormone-level trend received from the server device.

7. A computer-implemented method executed by at least one processor of a server device for predicting menstrual cycle fluctuations, comprising:
receiving menstrual-cycle–related health data including a cycle start date, basal body temperature values, and symptom log entries;
converting the received data into a normalized time-series feature vector;
retrieving historical cycle data associated with a user identifier;
constructing a structured prompt comprising labeled fields populated with the normalized time-series feature vector and the historical cycle data;
executing a transformer-based generative neural network model using the structured prompt to generate:
a predicted hormone-level fluctuation curve, and
a predicted next cycle start date;
generating a time-indexed recommendation schedule associated with the predicted hormone-level fluctuation curve;
transmitting the predicted hormone-level fluctuation curve and the time-indexed recommendation schedule to a user terminal device; and
updating a stored cycle-length estimation parameter using exponential smoothing based on a subsequently received actual cycle start date.

8. The method of claim 7, wherein converting includes scaling basal body temperature values to a standardized range between 0 and 1.

9. The method of claim 7, wherein constructing the structured prompt includes embedding cycle-phase tokens corresponding to follicular, ovulatory, and luteal phases.

10. The method of claim 7, further comprising generating a plurality of candidate hormone-level fluctuation curves and selecting one candidate based on a minimum forecast error metric.

11. The method of claim 7, wherein updating includes adjusting only a user-specific parameter layer without retraining the transformer-based generative neural network model.

12. The method of claim 7, wherein the time-indexed recommendation schedule includes a notification timing parameter for delivery to the user terminal device.

13. A non-transitory computer-readable storage medium storing instructions that, when executed by at least one processor of a server device, cause the at least one processor to:
receive menstrual-cycle–related health data formatted according to a predefined schema including timestamped basal body temperature and symptom entries;
generate a normalized time-series feature vector;
retrieve historical cycle data corresponding to a user identifier;
construct a structured prompt populated with the normalized time-series feature vector and the historical cycle data;
execute a transformer-based generative neural network model to generate:
a predicted hormone-level time-series dataset, and
a predicted cycle start date;
generate a time-indexed recommendation dataset corresponding to the predicted hormone-level time-series dataset;
transmit the predicted datasets to a user terminal device; and
update a stored cycle-length smoothing parameter using an exponential smoothing function based on a subsequently received actual cycle start date.

14. The non-transitory computer-readable storage medium of claim 13, wherein the predefined schema is a JSON-formatted structure including fields for user identifier, timestamp, temperature value, and symptom intensity score.

15. The non-transitory computer-readable storage medium of claim 13, wherein the transformer-based generative neural network model includes a self-attention mechanism configured to weight recent cycle data more heavily than older cycle data.

16. The non-transitory computer-readable storage medium of claim 13, wherein the instructions further cause the processor to calculate and transmit a confidence score associated with the predicted hormone-level time-series dataset.

17. The non-transitory computer-readable storage medium of claim 13, wherein updating comprises recalculating a predicted average cycle length parameter.

18. The non-transitory computer-readable storage medium of claim 13, wherein the instructions further cause the processor to log each predicted dataset and corresponding actual cycle start date for periodic offline retraining of the transformer-based generative neural network model.
